(19) United States
(12) Patent Application Publication
(10) Pub. No.: US 2019/0353724 A1
(43) Pub. Date: Nov. 21, 2019

(54) MAGNETIC RESONANCE ANTENNA COMPATIBLE WITH CHARGED PARTICLE ACCELERATOR SYSTEMS

(71) Applicant: KONINKLIJKE PHILIPS N.V., EINDHOVEN (NL)

(72) Inventor: JEROEN SNELTEN, Eindhoven (NL)

(21) Appl. No.: 16/469,973

(22) PCT Filed: Dec. 14, 2017
(86) PCT No.: PCT/EP2017/082934

§ 371 Date: Jun. 14, 2019

(30) Foreign Application Priority Data
Dec. 15, 2016 (EP) 16204232.9

(51) Int. Cl. G01R 33/36; G01R 33/48; A61B 5/00; A61B 5/055; A61N 5/10

(52) U.S. Cl. CPC G01R 33/3657; G01R 33/4808; A61N 2005/1055; A61B 5/055; A61N 5/1049; A61B 5/0036

(57) ABSTRACT

The invention provides for a medical instrument (100) comprising a magnetic resonance imaging system (104) with an imaging zone (132). The medical instrument further comprises an external beam radiotherapy system (102). A magnetic resonance antenna (129) surrounds the imaging zone. The magnetic resonance antenna comprises at least one detuning circuit (131) comprising at least one solid state switching element (308, 400, 900, 902) for switching the magnetic resonance antenna between a tuned mode and a detuned mode. The at least one solid state switching element conducts current in the detuned mode. The magnetic resonance antenna comprises at least one antenna element (300) comprising a tuning capacitor (302). The detuning circuit is connected in parallel with the tuning capacitor. The detuning circuit comprises a primary LC circuit (306) in series with the at least one solid state switching element.

### FIELD OF THE INVENTION

The invention relates to magnetic resonance imaging.

### BACKGROUND OF THE INVENTION

Integration of Magnetic Resonance Imaging (MRI) and Linear Accelerators (LINAC) opens new horizons in Radiotherapy by improved lesion targeting, especially for moving organs. In a practical implementation proposal, the LINAC rotates around the subject to hit the gross target volume (GTV) and clinical target volume (CTV) from multiple angles while minimizing the radiation exposure for surrounding tissues.

The combination of magnetic resonance apparatuses and LINAC radiotherapy sources is known. Typically a LINAC source is placed on a rotating gantry about the magnet and the magnet designed such that the LINAC rotates in a zero-field region of the magnet. Another particular feature of the concept is the use of a split gradient coil which prevents attenuation of the LINAC beam.

The journal article Lamey et. al., “Radio frequency shielding for a linac-MRI system,” Phys. Med. Biol. 55 (2010) 995-1006 doi: 10.1088/0031-9155/55/4/006 describes the advantages of shielding an MRI from a LINAC using radio frequency (RF) shielding.

### SUMMARY OF THE INVENTION

The invention provides for a medical instrument, a computer program product, and a method in the independent claims.

As disclosed in Lamey et. al., RF noise can be a problem when using a medical instrument that integrates MRI and LINAC systems. What is not discussed in this journal article is that often times the source of RF noise can be from the interaction between the MRI system and LINAC system. The MRI portion of the medical instrument is typically configured as a conventional MRI system. The MRI system will have a built in body coil or bird cage coil, which is referred to herein as a magnetic resonance antenna. The MRI system will also be capable of using specialized local coils that can also be used to image a subject. For example a smaller birdcage coil could be placed around a subjects head or a surface coil could be laid upon the subject. This supplementary coil is referred to as a, magnetic resonance coil. The terms “magnetic resonance antenna” and “magnetic resonance coil” are labels which are chosen to enable the reader to distinguish between two separate coils.

Often both the magnetic resonance antenna and the magnetic resonance coil are tuned to operate at the same frequency or frequencies. This means that when the magnetic resonance coil is used, it is beneficial to de-tune the magnetic resonance antenna so that the effect of coupling between the magnetic resonance antenna and the magnetic resonance coil is minimized. What is typically done is that a solid state switching element, such as a PIN diode is used to control detuning circuit to switch the magnetic resonance antenna between a tuned and an untuned state. What is currently done is that a voltage is supplied to the PIN diode to place the magnetic resonance antenna into a tuned state. The problem with this is that when the LINAC is operated, ionizing radiation can be scattered into the PIN diode. This may cause conduction or electron avalanches within the PIN diode, which then results in the magnetic resonance antenna producing RF noise that can interfere with the operation of the magnetic resonance coil.

Embodiments of the invention may reduce the amount of RF noise produced by the magnetic resonance antenna by modifying the detuning circuit such that the magnetic resonance antenna is in the detuned state when current is supplied to the PIN diode. If radiation is then scattered into PIN diode, it produces very little or essentially no RF noise. The PIN diode is already conducting current, any avalanche effect that happens within the PIN diodes produces a small or negligible chance of the current flowing through the PIN diode. There is no avalanche when a PIN diode is conducting in a forward state.

In one aspect, the invention provides for a medical instrument comprising a magnetic resonance imaging system with an imaging zone. The medical instrument further comprises an external beam radiotherapy system with a target zone. The target zone is within the imaging zone. The magnetic resonance imaging system may for instance be used for guiding the external beam radiotherapy system. The imaging of the magnetic resonance imaging system may be registered with respect to the coordinate system of the external beam radiotherapy system. The magnetic resonance imaging system may for example be used to determine the local anatomy of a subject placed within the imaging zone.

The medical instrument further comprises a magnetic resonance antenna surrounding the imaging zone. The magnetic resonance antenna comprises at least one detuning circuit. Each of the at least one detuning circuits comprises at least one solid state switching element for switching the magnetic resonance antenna between a tuned mode and a detuned mode. The solid state switching element is configured for conducting current in the detuned mode. The medical instrument further comprises a magnetic resonance coil configured for acquiring magnetic resonance data. Often times there may be more than one antenna or coil within a magnetic resonance imaging system. The magnetic resonance antenna and the magnetic resonance coil are both intended to be antennas or coils used in data acquisition during the acquisition of magnetic resonance data. The term magnetic resonance antenna and the term magnetic resonance coil is used to distinguish between these two distinct antenna or coil systems. The medical instrument further comprises a control source for supplying electrical current to the solid state switching element when the magnetic resonance antenna is in the detuned mode. The magnetic resonance antenna may be configured for transmitting and/or receiving in different examples.

This medical instrument may have the benefit of having reduced amounts of noise detected by the magnetic resonance coil when the magnetic resonance antenna is detuned. Solid state devices such as PIN diodes, FET transistors, or bi-polar transistors may sometimes generate electric noise when exposed to ionizing radiation. For example a external beam radiotherapy system may generate ionizing radiation which is scattered into the solid state switching elements. If the solid state switching element is configured such that it conducts current in the detuned mode then the magnetic resonance coil can be operated without a fear of noise being generated by the magnetic resonance antenna. For example if a magnetic resonance antenna were constructed differently such that the switching element was configured to be detuned when it is not being supplied with current then ionizing radiation may cause avalanches or breakdown within the solid state switching element and cause momentary bursts or amounts of noise which then could be picked up by the magnetic resonance coil. The medical instrument may therefore have the ability to measure magnetic resonance data with a reduced amount of noise using the magnetic resonance coil.

The magnetic resonance antenna and/or the magnetic resonance coil may be tuned for transmitting or receiving data at a magnetic resonance frequency. This frequency may for example be dependent upon the magnetic field of a magnet used to generate a magnetic field for a magnetic resonance imaging system and also the particular magnetic resonance that is of interest. In some examples the magnetic resonance antenna and/or the magnetic resonance coil may be tuned to multiple frequencies. For example the magnetic resonance imaging system may be configured for multi-nuclei frequency work. In this case the magnetic resonance antenna and/or the magnetic resonance coil may be tuned to one or more of these multiple frequencies.

In another embodiment, the medical instrument further comprises a memory for storing machine-executable instructions by a processor and also for storing pulse sequence commands. Pulse sequence commands as used herein encompass commands that may be used to directly control the magnetic resonance imaging system to acquire magnetic resonance data or it may be data which may be converted into such instructions. For example the pulse sequence commands may also be in the form of a timing chart or diagram which details which operations the magnetic resonance imaging system performs at a various time to acquire magnetic resonance data.

The medical instrument further comprises a processor for controlling the medical instrument. Execution of the machine-executable instructions further causes the processor to control the magnetic resonance imaging system with the pulse sequence commands to acquire the magnetic resonance data using the magnetic resonance coil. Execution of the machine-executable instructions further cause the processor to control the control source to supply current to the solid state switching element to place the magnetic resonance antenna into the detuned state during acquisition of magnetic resonance data using the magnetic resonance coil. In this case the control source supplies electrical current to the solid state switching element. This may be beneficial because even if ionizing radiation is scattered into the solid state switching element the solid state switching element is already conducting current. The magnetic resonance antenna may therefore generate less noise when the magnetic resonance coil is used in the acquisition of the magnetic resonance data.

In another embodiment, the magnetic resonance coil is a surface coil. For example the surface coil may be placed on a surface of the subject and used during the acquisition of the magnetic resonance data.

In another embodiment, the external beam radiotherapy system is a LINAC.

In another embodiment, execution of the machine-executable instructions further cause the processor to control the external beam radiotherapy system to irradiate at least a portion of the target zone during acquisition of the at least a portion of the magnetic resonance data.

In another embodiment, the magnetic resonance antenna comprises at least one antenna element. The at least one antenna element comprises a tuning capacitor. The detuning circuit is connected in parallel with the tuning capacitor. The detuning circuit comprises a primary LC circuit in series with the solid state switching element. The at least one antenna element may for example be rungs of a birdcage or surface coil. Attaching the detuning circuit across the tuning capacitor may provide for a convenient way of adding an element which enables the particular antenna element to be detuned. Placing the LC circuit and the solid state switching element in series may be beneficial because the LC circuit may filter noise generated by the at least one slid state switching element.

In another embodiment, the primary LC circuit and the at least one solid state switching element are connected in series across the tuning capacitor. This may be beneficial because the LC circuit will filter noise generated in the at least one solid state switching element.

In some examples, the primary LC circuit and also the later mentioned LC circuit may be a tank circuit. A tank circuit is at least an inductance and capacitance in parallel which has a resonance together with the tuning capacitor. In some examples the primary LC circuit and also the secondary LC circuit may also be tuned to more than one resonance. For example there may be particular magnetic resonance frequencies which are used during the acquisition of magnetic resonance data. The LC circuits may be tuned to these one or more frequencies together with the tuning capacitor.

In another embodiment, the tuning circuit is tuned to one or more multiple frequencies. These for example may be the magnetic resonance frequencies that are determined by the magnetic field of the magnetic resonance imaging system and the particular resonances of interest.

In another embodiment, the tuning circuit comprises a secondary LC circuit in series with the solid state switching element. The solid state switching element is between the primary LC circuit and the secondary LC circuit which is resonance on the MR frequency.

In another embodiment, the primary LC circuit and the secondary LC circuit have equal impedances. This and also the previous embodiment may have the benefit that the voltage drop across the solid state switching element may be more symmetric.

In another embodiment, the tuning circuit further comprises an passive switched filter element. The solid state switching element is between the primary LC circuit and the passive filter element. The use of a passive filter element may be beneficial because it may be useful in reducing noise produced by the magnetic resonance antenna in coil Receive mode so not in Body coil detune mode even further.

In another embodiment, the passive filter circuit comprises a PIN diode in parallel with a passive filter. The additional PIN diode may be useful in switching the passive filter circuit on and off when the Body coil is detuned.

In another embodiment, the solid state switching element comprises at least one PIN diode. The use of PIN diodes is well known and is known to be compatible in the magnetic resonance imaging system.

In another embodiment, the at least one PIN diode is connected in parallel with multiple high speed diodes. Putting a number of high speed diodes in parallel with a PIN diode may have the benefit of reducing the switching time of the at least one PIN diode.

In another embodiment, the solid state switching element is a FET transistor or a bi-polar transistor. When an FET transistor or bi-polar transistor is used the control source may also additionally apply a voltage source to switch the FET in addition to supplying the current.

In another embodiment, the magnetic resonance antenna is a birdcage coil.

In another embodiment, the magnetic resonance antenna is a body coil.

In another aspect, the invention provides for a method of operating the medical instrument. The medical instrument comprises a magnetic resonance imaging system with an imaging zone. The medical instrument further comprises a external beam radiotherapy system with a target zone. The target zone is within the imaging zone. The medical instrument further comprises a magnetic resonance antenna surrounding the imaging zone. The magnetic resonance antenna comprises at least one detuning circuit. Each of the at least detuning circuit comprises a solid state switching element for switching the magnetic resonance antenna between a tuned mode and a detuned mode.

The solid state switching element is configured for conducting current in the detuned mode. The medical instrument further comprises a control source for supplying electrical current to the solid state switching element when the magnetic resonance antenna is in the detuned mode. The medical instrument further comprises a magnetic resonance coil. The method comprises controlling the magnetic resonance imaging system with the pulse sequence commands to acquire magnetic resonance data using the magnetic resonance coil. The method further comprises controlling the control source to supply current to the solid state switching element to place the magnetic resonance antenna into the tuned state when an acquisition of the magnetic resonance data using the magnetic resonance coil is performed.

In another aspect, the invention provides for a computer program product for execution by a processor controlling a medical instrument. The computer program product comprises machine-executable instructions. The medical instrument comprises a magnetic resonance imaging system with an imaging zone. The medical instrument further comprises a external beam radiotherapy system with a target zone. The target zone is within the imaging zone. The medical instrument further comprises a magnetic resonance antenna surrounding the imaging zone. The magnetic resonance antenna comprises at least one detuning circuit. Each of the at least one detuning circuit comprises a solid state switching element for switching the magnetic resonance antenna between the tuned mode and a detuned mode. The solid state switching element is configured for conducting current in the detuned mode. The medical instrument further comprises a control source for supplying electrical current to the solid state switching element when the magnetic resonance antenna is in the detuned mode. The medical instrument further comprises a magnetic resonance coil.

Execution of the machine-executable instructions cause the processor to control the magnetic resonance imaging system with the pulse sequence commands to acquire the magnetic resonance data using the magnetic resonance coil. Execution of the machine-executable instructions further cause the processor to control the control source to supply current to the solid state switching element to place the magnetic resonance antenna into the tuned state during acquisition of the magnetic resonance data using the magnetic resonance coil.

It is understood, that one or more of the aforementioned embodiments of the invention may be combined as long as the combined embodiments are not mutually exclusive.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example, a data may be retrieved over a modem, over the internet, or over a local area network. Computer executable code embodied on a computer readable medium may be transmitted using any appropriate medium, including but not limited to wireless, wire line, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. Computer memory may be any volatile or non-volatile computer-readable storage medium.

A ‘processor’ as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object oriented programming language such as Java, Smalltalk, C++ or the like and conventional procedural programming languages, such as the C programming language or similar programming languages and compiled into machine executable instructions. In some instances the computer executable code may be in the form of a high level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It is understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, pedals, wired glove, remote control, and accelerometer are all examples of user interface components which enable the receiving of information or data from an operator.

A ‘hardware interface’ as used herein encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, bluetooth connection, wireless local area network connection, TCP/IP connection, ethernet connection, control voltage interface, MIDI interface, analog input interface, and digital input interface.

Magnetic Resonance (MR) data is defined herein as being the recorded measurements of radio frequency signals emitted by atomic spins using the antenna of a magnetic resonance apparatus during a magnetic resonance imaging scan. Magnetic resonance data is an example of medical imaging data. A Magnetic Resonance (MR) image is defined herein as being the reconstructed two or three dimensional visualization of anatomic data contained within the magnetic resonance imaging data.

### BRIEF DESCRIPTION OF THE DRAWINGS

In the following preferred embodiments of the invention will be described, by way of example only, and with reference to the drawings in which:

FIG. 1 illustrates an example of a medical instrument;

FIG. 2 shows a flow chart which illustrates an example of a method of operating the medical instrument of FIG. 1;

FIG. 3 illustrates an example of a detuning circuit;

FIG. 4 illustrates a further example of a detuning circuit;

FIG. 5 illustrates a further example of a detuning circuit;

FIG. 6 illustrates a further example of a detuning circuit;

FIG. 7 illustrates a further example of a detuning circuit;

FIG. 8 shows a time vs. current plot for a PIN diode;

FIG. 9 shows a combination of normal PIN diodes and high speed diodes; and

FIG. 10 is a plot used to describe a self-biasing effect in the combination of FIG. 9.

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

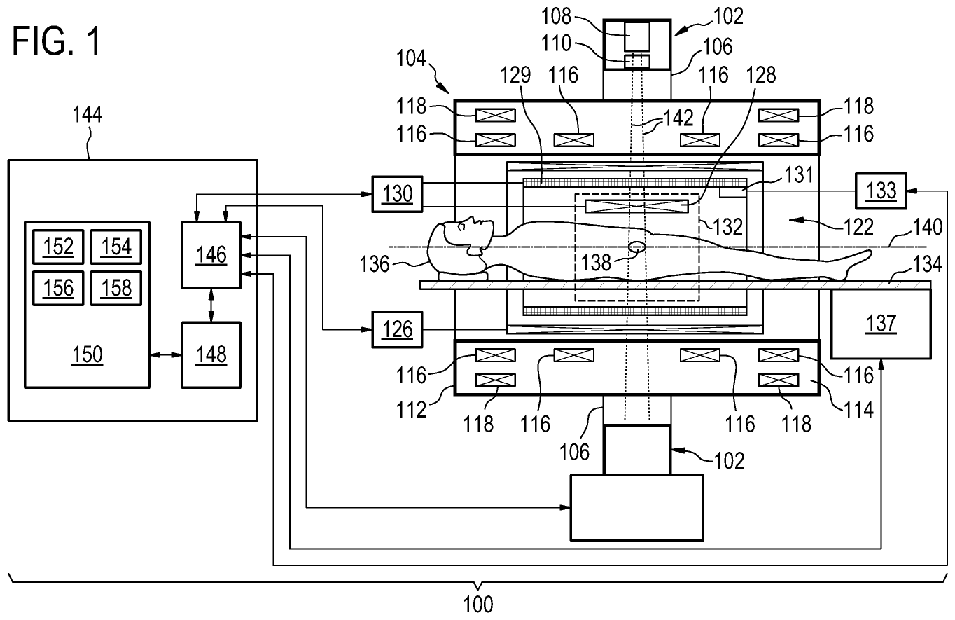

FIG. 1 illustrates an example of a medical instrument 100. The medical instrument 100 comprises a external beam radiotherapy system 102 and a magnetic resonance imaging system 104. The external beam radiotherapy system 102 comprises a gantry 106 and a radiotherapy source 108. The gantry 106 is for rotating the radiotherapy source 108 about an axis of gantry rotation 140. Adjacent to the radiotherapy source 108 is a collimator 110. The magnetic resonance imaging system 104 comprises a magnet 112.

It is also possible to use permanent or resistive magnets. The use of different types of magnets is also possible for instance it is also possible to use both a split cylindrical magnet and a so called open magnet. A split cylindrical magnet is similar to a standard cylindrical magnet, except that the cryostat has been split into two sections to allow access to the iso-plane of the magnet, such magnets may for instance be used in conjunction with charged particle beam therapy. An open magnet has two magnet sections, one above the other with a space in-between that is large enough to receive a subject: the arrangement of the two sections area similar to that of a Helmholtz coil. Open magnets are popular, because the subject is less confined. Inside the cryostat of the cylindrical magnet there is a collection of superconducting coils. Within the bore of the cylindrical magnet there is an imaging zone where the magnetic field is strong and uniform enough to perform magnetic resonance imaging.

The magnet 112 shown in this embodiment is a standard cylindrical superconducting magnet. The magnet 112 has a cryostat 114 with superconducting coils within it 116. There are also superconducting shield coils 118 within the cryostat also. The magnet 112 has a bore 122.

Within the bore of the magnet is a magnetic field gradient coil 124 for acquisition of magnetic resonance data to spatially encode magnetic spins within an imaging zone of the magnet. The magnetic field gradient coil 124 is connected to a magnetic field gradient coil power supply 126. The magnetic field gradient coil 124 is intended to be representative, to allow radiation to pass through without being attenuated it will normally be a split-coil design. Typically magnetic field gradient coils contain three separate sets of coils for spatially encoding in three orthogonal spatial directions. The magnetic field gradient power supply 126 supplies current to the magnetic field gradient coils. The current supplied to the magnetic field coils is controlled as a function of time and may be ramped or pulsed.

There is a magnetic resonance coil 128 connected to a transceiver 130. The magnetic resonance coil 128 is adjacent to an imaging zone 132 of the magnet 112. The imaging zone 132 has a region of high magnetic field and homogeneity which is sufficient for performing magnetic resonance imaging. The magnetic resonance coil 128 may is for manipulating the orientations of magnetic spins within the imaging zone and for receiving radio transmissions from spins also within the imaging zone. The magnetic resonance coil 128 may also be referred to as an antenna or channel. The magnetic resonance coil 128 may contain multiple coil elements. The radio frequency antenna may also be referred to as a channel.

The magnetic resonance coil 128 and radio frequency transceiver 130 may be replaced by separate transmit and receive coils and a separate transmitter and receiver. It is understood that the magnetic resonance coil and the radio frequency transceiver are representative. The radio frequency antenna is intended to also represent a dedicated transmit antenna and a dedicated receive antenna. Likewise the transceiver may also represent a separate transmitter and receivers.

Also within the bore of the magnet 122 is a subject support 134 for supporting a subject 136. The subject support 134 may be positioned by a mechanical positioning system 137. Within the subject 136 there is a target zone 138. The axis of gantry rotation 140 is coaxial in this particular embodiment with the cylindrical axis of the magnet 112. The subject support 134 has been positioned such that the target zone 138 lies on the axis 140 of gantry rotation. The radiation source 108 is shown as generating a radiation beam 142 which passes through the collimator 303 and through the target zone 138. As the radiation source 108 is rotated about the axis 140 the target zone 138 will always be targeted by the radiation beam 142. The radiation beam 142 passes through the cryostat 114 of the magnet. The magnetic field gradient coil may have a gap which separate the magnetic field gradient coil into two sections. If present, this gap reduces attenuation of the radiation beam 142 by the magnetic field gradient coil 124. In some embodiments the magnetic resonance coil 128 may also have gaps or be separated to reduce attenuation of the radiation beam 142.

It can be seen that within the bore 122 of the magnet 112 there is a magnetic resonance antenna 129. In this example the magnetic resonance antenna 129 is a body coil. The magnetic resonance antenna 129 may have individual rungs or rods which have each individual detuning circuits 131. Only one detuning circuit 131 is shown in this Fig. Each of the detuning circuits 131 is connected to a control source 133. The control source comprises at least a current source and in some instances a voltage source additionally which is used for switching the detuning circuit into a tuned and detuned mode. The detuning circuit 131 illustrated in this Fig. goes into a detuned state when current is supplied by the control source 133 to the detuning circuit 131. Each of the detuning circuits comprises a solid state switching element that is configured for conducting current in the detuned mode. When ionizing radiation 142 is present scattered radiation may enter the solid state switching element in the detuning circuit 131. Having current already conducting reduces the chance that noise will be generated by the magnetic resonance antenna 129 that is then picked up by the radio-frequency coil 128.

The transceiver 130, the magnetic field gradient coil power supply 126, the control source, and the mechanical positioning system 137 are all shown as being connected to a hardware interface 146 of a computer system 144. The computer system 144 is shown as further comprising a processor 148 for executing machine executable instructions and for controlling the operation and function of the therapeutic apparatus. The hardware interface 146 enables the processor 148 to interact with and control the medical instrument 100. The processor 148 is shown as further being connected to a computer memory 150.

The computer memory 150 is shown as containing machine-executable instructions 152 which enable the processor 148 to control the operation and function of the various components of the medical instrument 100. The computer memory 150 is further shown as containing pulse sequence commands 154, which enable the processor 148 to control the magnetic resonance imaging system 104 to acquire magnetic resonance data. The computer memory 150 is further shown as containing magnetic resonance data 156 that was acquired by controlling the magnetic resonance imaging system 104 with the pulse sequence commands 154. The computer memory 150 is further shown as containing the magnetic resonance image 158 that was reconstructed from the magnetic resonance data 156. The magnetic resonance image 158 may for example be used to guide radiotherapy using the radiotherapy system 102.

It is not shown in this example, however there may be additional data and software components in the computer memory 150. For example there may be radiotherapy planning modules 478 that may use the magnetic resonance image 158 for guiding the target zone 138 to be properly positioned within the subject 136.

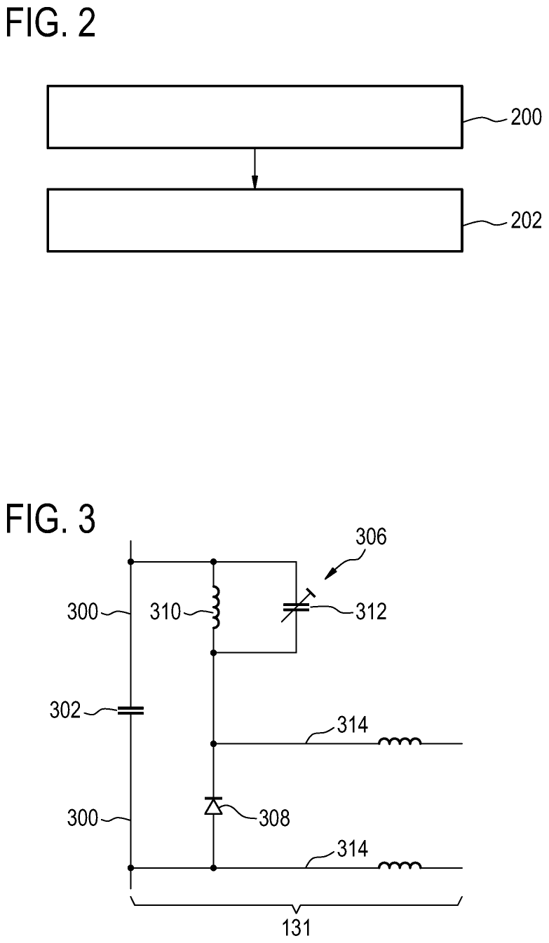

FIG. 2 shows a flowchart which illustrates a method of operating the medical instrument 100 shown in FIG. 1. First in step 100 the processor 148 controls the magnetic resonance imaging system 104 with the pulse sequence commands 154 to acquire the magnetic resonance data 156 using the radio-frequency coil 128. Next in step 102 the processor 148 controls the control source 133 to supply current to the solid state switching unit of the detuning circuit 131 to place the magnetic resonance antenna into the detuned state during acquisition of the magnetic resonance data 156 using the magnetic resonance coil 128.

Existing LINAC and MR combinations suffers from noise produced by a Body coil and disturbs the signal to noise ratio of a Receive coil used in the Body coil. During MR Linac operation both a receive coil inside the Body coil and radiation source are active. Since radiation has influence on the existing electronics in the Body coil, the noise level in the Body coil increases considerable. Due to E and B field coupling between Body coil and receiving coil inside the Body coil, noise is injected in the receive coil disturbing the signal to noise ratio which affects the diagnostic value of the image negatively.

Known methods produce noise during reception with receive coils inside a Body coil. The noise generated during Body coil detune in the existing situation is fully able to flow in the Body coil since the switching element (RF switching diode) that generates the noise is directly connected to the Body coil rods.

Several examples Cases A through F are discussed below:

Case A. Noise during body coil in Detune phase as is the original problem is not generated anymore due to Body coil detune with current instead of voltage. So the original disadvantage has been solved.

Now during Body coil Tune the switching element is producing noise. This not very critical but need attention in this description of the invention. Due to the basic character of the invention starting from Case B. below the signal to noise ratio degradation of the receiving Body coil is less.

FIG. 3 shows an example of the rods of a body coil 300 with a tuning capacitor 302. Across the tuning capacitor 302 there is a detuning circuit 131 attached. In this example the detuning circuit 131 comprises a primary LC circuit 306 in series with a PIN diode. The tune capacitor 302 in the body coil configuration can also be replaced with an Inductor that is resonant with 306. The primary LC circuit 306 in this example comprises an inductance 310 and a capacitance 312. The inductance 310 and capacitance 312 in parallel form a so called tank circuit which may be tuned to a particular frequency. In making the detuned circuit the tank circuit could for example be tuned to a particular magnetic resonance frequency that is wished to be specifically detuned from the magnetic resonance antenna, the LC of the tank circuit together with the tuning capacitor 302 of the body coil is resonant. The primary LC circuit 306 may however be comprised of more than just one inductor and capacitor 312 in parallel. There may for example be a more complicated impedance elements that have resonances at multiple peaks. In this case these multiple peaks may be each tuned to particular magnetic resonance frequencies that are being examined in the magnetic resonance imaging system.

The PIN diode 308 is used to switch between the tuned and detuned state. The lines 131 may for example be heavily or high RF impedance conductors that go to the control source. The control source in this example could be a current source with current limitation by a resistor. When the current source is used to supply forward current through the diode 308 the primary LC circuit 306 is effectively connected across the tuning capacitor 302 and the magnetic resonance antenna enters into the detuned state. In a tuned state a voltage is applied to the cathode of the PIN diode 308.

An addition here is the fact that multi nuclei coils that may have a different frequency than the main MR system frequency are used. Also for multi nuclei coils, used inside the quadrature body coil (QBC) (or magnetic resonance antenna), a sufficiently high QBC decoupling may be beneficial. The LC circuit that is active during QBC detune can be modified to a LC resonator circuit with more than one impedance maximum. For example one for the main MR frequency and one for a multi nuclei frequency. This results in a QBC that is decoupled for more than one frequency. Benefit here is better image quality over the volume of the QBC for the used frequencies.

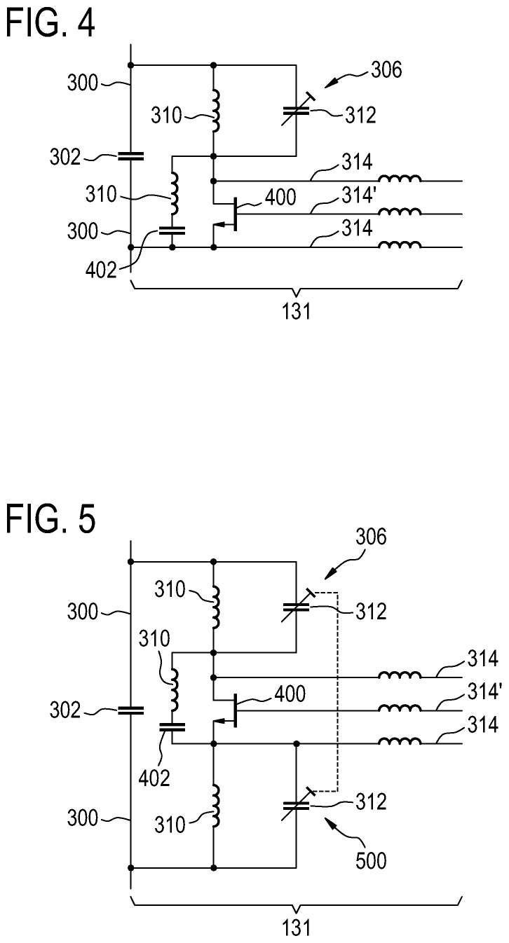

FIG. 4 shows a further example of a detuning circuit 131. The detuning circuit 131 in FIG. 4 is similar to FIG. 3. However in this example instead of a PIN diode an FET 400 transistor is used. The PIN diode has been replaced by a FET transistor 400 in parallel with an inductor 310. The lines 314 are again used to supply current by the control source. There is an additional control line 314′ that is used to supply a voltage to the gate of the FET 400. When current and voltage are supplied by the control source the FET transistor 400 effectively connects the primary LC circuit 306 across the tuning capacitor 302 and places the magnetic resonance antenna into the detuned state. The example shown in FIG. 4 may also be appropriately modified for use with a bi-polar transistor also.

Due to the fact a FET has a relatively large Drain capacitance and inductor has to be used to tune out this capacitance. The capacitance seen on the Drain of the FET exists of the sum of CDS, CDG and indirectly CGS. The inductor L tunes out all of these capacitances resulting in a FET switch that has relatively high impedance for the MR frequencies.

Due to the fact the circuit is asymmetric around the Tune capacitance the RF voltage on the Source terminal of the FET is relatively high which may cause a relatively high Source Gate voltage. The FET has to be able to withstand this voltage. A RF Gate Source voltage clamp circuit may be required. Due to the fact the DS source voltage across the FET during QBC Receive is relatively low the increase of noise due to radiation is also low which is beneficial.

FIG. 5 shows a modification of the detuning circuit 131. The detuning circuit 131 of FIG. 5 is similar to the detuning circuit of FIG. 4. However, in this example the primary LC circuit of FIG. 4 has been split into a primary 306 and a secondary LC circuit 500. The tune capacitor 302 in the body coil configuration can also be replaced with an inductor that is resonant with the capacitance of 306 and 500. The FET transistor 400 which functions as the solid state switching element is placed in series between the primary LC circuit and the secondary LC circuit 500. The term primary and secondary when referring to the LC circuits does not imply that one circuit is more important than the other, they are simply used as labels to distinguish between the two of them. The circuit in FIG. 5 can be constructed such that the impedance of the primary LC circuit 306 and the secondary LC circuit 500 can be identical. This may then have the benefit that the voltage 314′ and current leads 314 supplied by the control source are now fed to a virtual ground. This makes it easier to drive the solid state switching element or FET 400 in this case.

A potential benefit to the circuit of FIG. 5 is that detune and tune DC signals are fed to a virtual ground point causing low amplitude of RF signals to the DC driver connections that sources forward current and reverse voltage for the PIN diode. Filtering towards the DC sources is less critical. Also, clamping of GS or BE of the transistor can be simplified. When one uses a balanced configuration the virtual ground needs less GS or BE voltage, so less clamping is needed. The adjustable capacitors can be adjusted to be more or less the same value to keep balance. The adjustable capacitors may also be fixed capacitors.

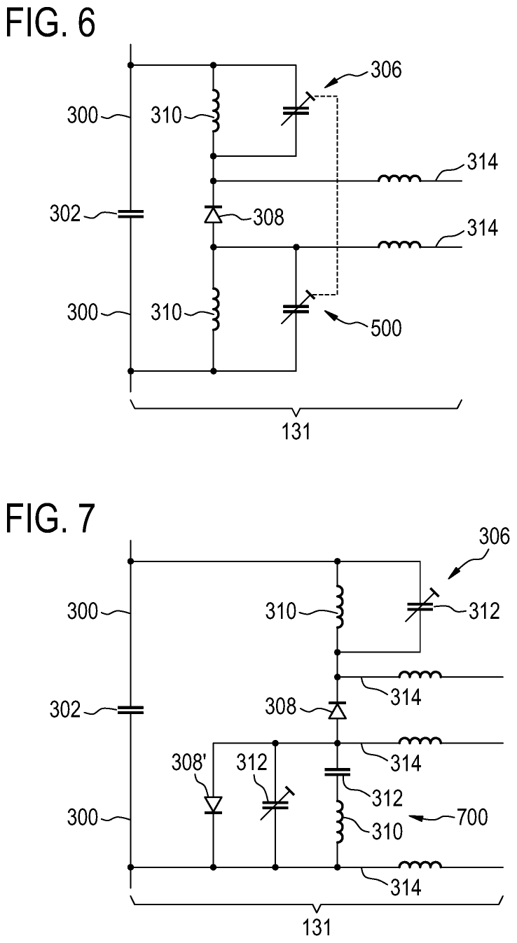

FIG. 6 shows a modification of the detuning circuit 131 of FIG. 3. In FIG. 6 the primary LC circuit 306 has been divided into a primary LC circuit 306 and a secondary LC circuit 500. Again, as with FIG. 5 the impedances of the primary LC circuit 306 and the secondary LC circuit 500 may be matched such that the PIN diode 308 sits at a virtual ground. This then makes it easier to drive the current 308 through the PIN diode 308 to control the detuning circuit 131.

In FIGS. 3 through 6 one can see the noise generated in the switching element is filtered by the resonance L and C, which is not resonant at the MR frequency, on top of the switching diode resulting is less noise in the Body coil so less noise coupled into the receive coil in the Body coil. This improves the noise generated in Body coil in receive. During Body coil in Transmit the noise level of the switching element is relatively low with respect to the Transmit power so not influencing the image quality result. A further improvement is described in Case B below.

Case B. In addition to the basic example given in Case A. A second possible implementation which improve the noise behavior of the Body coil in Receive if Case A. is not sufficiently suppressing the noise level during Body coil in receive mode disturbing the signal to noise ratio of the Body coil. Case B is illustrated in FIG. 7 below.

FIG. 7 shows a further modification of the detuning circuit 131 of FIG. 3. In this example, the components of the detuning circuit 131 in FIG. 3 are placed in series with an passive filter 700. The passive filter comprises an additional PIN diode 308′ that is connected in parallel with the trim capacitor 312 and a combination of an inductor and a capacitor 312 that are in series. The LC circuit in parallel with the PIN diode 308′ is tuned to the MR frequency so that no noise is injected from the RF switching diode on top of the LC diode circuit of the passive filter 700. The lines 314 are set to either different voltages or currents in order to place the magnetic resonance antenna into a tuned state, that may either be set for transmit or receive and a detuned state. The following table details how the circuit 131 of FIG. 7 can be operated.

| | States in FIG. 3 | | | | | |
|---|---|---|---|---|---|---|
| | QBC in Transmit state | | QBC in Receive state | | QBC in Detune state | |
| Voltage | Switching | | Switching | | Switching | |
| or current | PIN diode | Diode | PIN diode | Diode | PIN diode | Diode |
| active | 308 | 308′ | 308 | 308′ | 308 | 308′ |
| Reverse | Y | N | Y | 0 V *) | N | N |
| voltage | | | | | | |
| Forward | N | Y | N | N | Y | Y |
| current | | | | | | |

Note:
Diode D may be a PIN diode.
*) The Capacitance of D at 0 V (or at a relatively low voltage) is parallel resonant with capacitor and inductance connected to the anode of RF switching PIN.
308′, 312, 312 and 310 are together parallel resonant at the MR frequency.

In the above describe examples with FET's the FET may be replaced by a bipolar transistor.

Case B further suppresses the noise generated by the Body coil during the Body coil Receive phase and LINAC radiation source active. This has been achieved by a switched noise filter that is active in Body coil Tune (Receive). The main feature of the extension of example Case A. is that a noise filter is added that copes with both Body coil Transmit and Receive phases. In the Body coil receive phase the voltage across the filter switching element diode 308′ is zero. The capacitance of 308′ is part of the parallel resonator together with T and L and tuned on f0 by trimmer T. During Body coil Transmit, 308′ is fully conducting by a current sources, connecting the anode of the RF switching (PIN) diode to the lower part of the Tune capacitance of the Body coil. This has been done to be sure the DC reverse voltage applied is fully across the RF switching diode. Also no current will flow in the parallel resonant circuit during Body coil Transmit phase. In fact the leakage current through the RF switching diode during the Body coil Transmit phase will become Q times higher in the parallel resonant circuit which should result in unpredictable behavior of the circuit when 308′ is not driven in conduction by a current source. The leakage current of the RF switching diode is usually very small and not sufficient to open 308′ during Body coil Receive. In case of a relatively high leakage current of the RF switching diode a resistor R can be added which has a high value to by-pass the leakage current. The value can be 100 kΩ so not influencing the quality of the noise filter.

Generally, noise produced in a switching semiconductor can be filtered. Across the RF decoupling inductors a capacitor may be placed to improve isolation to the power supplies. The capacitors in parallel to the inductors form a parallel resonant circuit with high impedance.

Lowering the reverse voltages of the switching diode to zero V means no noise current is produced by the diode but also that the body coil is not well detuned. Coils used in the Body coil face more losses and frequency shift. When the coil used in the Body coil is a Transmit Receive coil and making the reverse voltage across the diode low or zero non-linear distortion of the magnetic field (B-field) will occur which is influencing the image quality negatively. Also Local SAR may be exceeded due to coupling. In fact a magnetic field probe (B-field probe) in a MR scanner is calibrated in a uniform B-field. Local B-field increase due to coupling with a second coil may not be detected by this flux probe. This allows the situation of an unwanted SAR level while using a Transmit Receive coil in the Body coil.

Distortion of the B1 field and unexpected B1 or E1 field distribution may influence the image quality and affect patient safety in terms of the specific absorption ratio (SAR). Also the non-linear distortion due to the non-biased semiconductors (diodes) may result in degraded image quality. For example, third order mixing products that will be in the MR receiver bandwidth affect the image quality.

Case D. FIG. 7 can be further simplified to a configuration where no reverse voltage is applied to the RF switching diode. This may have some disadvantages. The advantage is clear that no DC voltage is needed.

Since the reverse DC high voltage across the RF switching diode is mainly necessary during Body coil Transmit phase (and may be lowered during Body coil Receive phase)

During Body coil Transmit the RF switching diode which needs to be well reverse biased can be used as rectifier diode to generate his own reverse voltage. This is called self-biasing. The challenge here is that the diode needs to conduct in a number of RF periods when the RF Transmit pulse is applied. Usually a PIN diode is used as RF switching element which is a not very fast rectifier. Relative high power dissipation in the RF switching diode may occur during to the build-up of the reverse voltage. Choosing the right carrier lifetime of the PIN diode may help here to limit the power dissipation during self-biasing. Another challenge is that for very short RF pulses the Body coil is not tuned well.

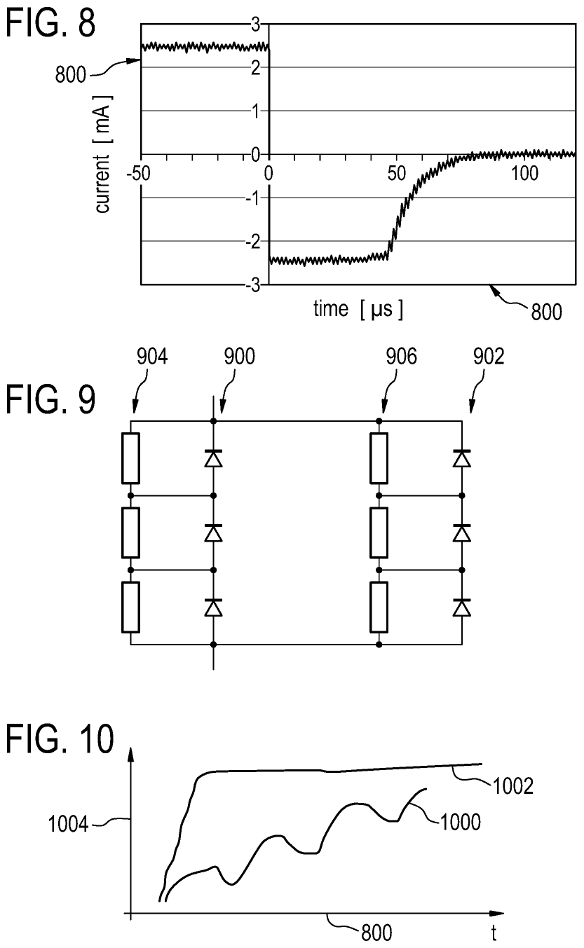

Case E. The disadvantages of Case D may be lowered by using so called speed up diodes in parallel to the relatively slow PIN diode. An example is given in FIG. 8. The high speed self-biasing diodes are much faster than the RF switching diodes so taking over the self-biasing for the first number of RF cycles so preventing the PIN diodes to heat up too much. In FIG. 7 the PIN diodes are switched in series which is basically not necessary but has been done to be able to take lower voltage PIN diodes that are cheaper and has the benefit of lower total leakage current.

FIG. 8 is used to illustrate the switching behavior of a PIN diode. The x-axis is the time in microseconds and the x-axis is the current in mA 802. At t=0 reverse biasing starts. As you can see the reverse current is not zero very quickly. This is caused by the fact the I-layer of the P I N diode contains carriers that have to be removed from the I layer before the PIN diode reverse current is low.

FIG. 9 shows an arrangement which may be used to speed up the reaction of the PIN diodes by providing for self-biasing. In FIG. 9 there is a parallel circuit with two main branches. First there are one or more PIN diodes 900 in series. Regular PIN diodes provide a high breakdown of voltage. In parallel with the one or more PIN diodes in series 900 there is additionally a chain of high speed diodes 902 in series. The use of the high speed diodes enables a reduction in the time for the PIN diodes 900 to recover when reversing the PIN diodes. The breakdown voltage 902 of the high speed diodes is not as large as the PIN diodes 900. Typically for each of the PIN diodes 900 there will be a number of high speed diodes 902. There is additionally a voltage divider comprising for resistors that are connected to the PIN diodes 900 and another voltage divider 906 attached across the high speed diodes 902. This is to ensure that the voltage drop across each of the PIN diodes 900 and across each of the high speed diodes 902 remains constant. The circuit arrangement shown in FIG. 9 may be used to replace each PIN diode 308 or 308′ shown in the previous Figs.

FIG. 10 shows a plot of time 800 versus voltage 1004 to illustrate a self-biasing effect in the arrangement of FIG. 9. Curve 1000 shows the cathode voltage self-biasing of the PIN diodes. Curve 1002 shows the cathode voltage of the high-speed PIN diodes. The link with the efficiency of self-biasing is that a positive sinusoidal voltage charges the capacitor in the top voltage rectifier example. When the next sine wave part becomes negative some of the charge in the capacitors leaks back to the source because the carrier lifetime. This process causes power dissipation in the PIN diode. PIN diodes with relative short carrier life time suffer less from high power dissipation during self-biasing but may have relatively high On-resistance. The speed-up diodes take care for the quick build-up of the cathode voltage such that the PIN diode does not need to do this.

Case F. In fact Case E can be considered to be a kind of passive detuning.

Examples of Case A thorough F may have one or more of the following features:

Main: Not to generate noise in the Body coil in Detune that couples into the receiving coil positioned into the Body coil.

Not to generated noise in the Body coil in Detune that couples into the receiving coil positioned into the Body coil. Not to generate noise in Body coil in Tune that disturbs the signal to noise ratio of the Body coil itself.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE NUMERALS

100 medical instrument
102 external beam radiotherpay system
104 magnetic resonance imaging system
106 gantry
108 radiotherapy source
110 collimator
112 magnet
114 cryostat
116 superconducting coil
118 superconducting shield coil
122 bore
124 magnetic field gradient coil
126 magnetic field gradient coil power supply
128 magnetic resonance coil
129 magnetic resonance antenna
130 transciever
131 detuning circuit
132 imaging zone
133 control source
134 subject support
136 subject
137 mechanical positioning system
138 target zone
140 axis of gantry rotation
142 radiation beam
144 computer system
146 hardware interface
148 processor
150 computer memory
152 machine executable instructions
154 pulse sequence commands
156 magnetic resonance data
158 magnetic resonance image
200 control the magnetic resonance imaging system with the pulse sequence commands to acquire the magnetic resonance data using the magnetic resonance coil
202 control the control source to supply current to the solid state switching element to place the magnetic resonance antenna into the detuned state during acquisition of the magnetic resonance data using the magnetic resonance coil
300 rod of body coil
302 tuning capacitor
306 primary LC circuit
308 PIN diode
308′ PIN diode
310 inductance
312 DC blocking capacitance
314 to control source (current source)
314′ to control source (voltage source)
400 FET
500 secondary LC circuit
700 passive filter
800 time
802 current
900 PIN diodes in series
902 high speed diodes in series
904 voltage divider
906 voltage divider
1000 cathode voltage self-biasing pin diodes
1002 cathode voltage due to high speed diodes
1004 time

1. A medical instrument comprising:
a magnetic resonance imaging system with an imaging zone;
a magnetic resonance antenna surrounding the imaging zone, wherein the magnetic resonance antenna comprises at least one detuning circuit, wherein each of the at least one detuning circuit comprises at least one solid state switching element for switching the magnetic resonance antenna between a tuned mode and a detuned mode, wherein the at least one solid state switching element is configured for conducting current in the detuned mode, wherein the magnetic resonance antenna comprises at least one antenna element, wherein the at least one antenna element comprises a tuning capacitor, wherein the detuning circuit is connected in parallel with the tuning capacitor, wherein the detuning circuit comprises a primary LC circuit in series with the at least one solid state switching element;
a magnetic resonance coil configured for acquiring magnetic resonance data; and
a control source for supplying electrical current to the at least one solid state switching element when the magnetic resonance antenna is in the detuned mode.

2. The medical instrument of claim 1, wherein the medical instrument further comprises:
a processor for controlling the medical instrument;
a memory for storing machine executable instructions for execution by the processor and pulse sequence commands;
wherein execution of the machine executable instructions causes the processor to:
control the magnetic resonance imaging system with the pulse sequence commands to acquire the magnetic resonance data using the magnetic resonance coil; and
control the control source to supply current to the at least one solid state switching element to place the magnetic resonance antenna into the detuned state during acquisition of the magnetic resonance data using the magnetic resonance coil.

3. The medical instrument of claim 2, wherein execution of the machine executable instructions further causes the processor to control the external beam radiotherapy system to irradiate at least a portion of the target zone during acquisition of the at least a portion of the magnetic resonance data.

4. The medical instrument of claim 1, wherein the detuning circuit is tuned to one or more frequencies.

5. The medical instrument of claim 4, wherein the detuning circuit comprises a secondary LC circuit in series with the at least one solid state switching element, wherein the at least one solid state switching element is between the primary LC circuit and the secondary LC circuit.

6. The medical instrument of claim 5, wherein the primary LC circuit and the secondary LC circuit have equal impedances.

7. The medical instrument of claim 4, wherein the tuning circuit further comprises an passive switched filter element, wherein the at least one solid state switching element is in parallel to the secondary LC circuit and the passive switched filter element.

8. The medical instrument of claim 7, wherein the active passive circuit comprises a diode in parallel with a passive filter.

9. The medical instrument of claim 1, wherein the at least one solid state switching element comprises at least one diode.

10. The medical instrument of claim 9, wherein the at least one diode is connected in parallel with multiple high speed diodes.

11. The medical instrument of claim 1, wherein the at least one solid state switching element is a FET transistor or a bi-polar transistor.

12. The medical instrument of claim 1, wherein the magnetic resonance antenna is a bird cage coil or a body coil.

13. The medical instrument of claim 1, wherein the primary LC circuit and the at least one solid state switching element are connected in series across the tuning capacitor.

14. A method of operating a medical instrument, wherein the medical instrument comprises a magnetic resonance imaging system with an imaging zone, wherein the medical instrument further comprises a external beam radiotherapy system with a target zone, wherein the target zone is within the imaging zone; wherein the medical instrument further comprises a magnetic resonance antenna surrounding the imaging zone, wherein the magnetic resonance antenna comprises at least one detuning circuit, wherein each of the at least one detuning circuit comprises at least one solid state switching element for switching the magnetic resonance antenna between a tuned mode and a detuned mode, wherein the at least one solid state switching element is configured for conducting current in the detuned mode, wherein the medical instrument further comprises a control source for supplying electrical current to the at least one solid state switching element when the magnetic resonance antenna is in the detuned mode, wherein the magnetic resonance antenna comprises at least one antenna element, wherein the at least one antenna element comprises a tuning capacitor, wherein the detuning circuit is connected in parallel with the tuning capacitor, wherein the detuning circuit comprises a primary LC circuit in series with the at least one solid state switching element, wherein the medical instrument further comprises a magnetic resonance coil,
wherein the method comprises:
controlling the magnetic resonance imaging system with pulse sequence commands to acquire magnetic resonance data using the magnetic resonance coil; and
controlling the control source to supply current to the at least one solid state switching element to place the magnetic resonance antenna into the detuned state during acquisition of the magnetic resonance data using the magnetic resonance coil.

15. (canceled)

16. The medical instrument of claim 1, further comprising a radiotherapy system with a target zone, wherein the target zone is within the imaging zone.

17. The medical instrument of claim 1, wherein the medical instrument is compatible with an external beam radiotherapy system having a target zone, wherein the target zone is within the imaging zone.

18. A magnetic resonance apparatus, comprising:
a magnetic resonance antenna surrounding an imaging zone, wherein the magnetic resonance antenna comprises at least one detuning circuit and at least one antenna element,
wherein each of the at least one detuning circuit comprises at least one solid state switching element for switching the magnetic resonance antenna between a tuned mode and a detuned mode,
wherein the at least one solid state switching element is configured for conducting current in the detuned mode,
wherein the at least one antenna element comprises a tuning capacitor,
wherein the detuning circuit is connected in parallel with the tuning capacitor,
wherein the detuning circuit comprises a primary LC circuit in series with the at least one solid state switching element.

19. The magnetic resonance antenna apparatus of claim 18, wherein the at least one solid state switching element is configured to receive electrical current from a control source when the magnetic resonance antenna is in the detuned mode.

20. The magnetic resonance antenna apparatus of claim 18, wherein the apparatus is included in a medical instrument comprising a magnetic resonance imaging system with an imaging zone.

21. The magnetic resonance antenna apparatus of claim 20, wherein the medical instrument includes a radiotherapy system having a target zone.
